(19) United States
(12) Patent Application Publication
(10) Pub. No.: US 2024/0399322 A1
(43) Pub. Date: Dec. 5, 2024

(54) METHOD AND SYSTEM FOR PREPARING A BIOMATERIAL

(71) Applicant: BIOMIMETIC INNOVATIONS LIMITED, SHANNON, CO. CLARE (IE)

(72) Inventor: Philip PROCTER, Divonne le Bains (FR)

(21) Appl. No.: 18/699,652

(22) PCT Filed: Oct. 10, 2022
(86) PCT No.: PCT/EP2022/078072

§ 371 Date: Apr. 9, 2024

(30) Foreign Application Priority Data
Oct. 11, 2021 (SE) 2151247-0

(51) Int. Cl. B01F 23/50; B01F 31/80; B01F 31/86; B01F 35/21; B01F 35/22; B01F 35/221; B01F 101/22

(52) U.S. Cl. CPC B01F 23/51; B01F 23/551; B01F 31/86; B01F 31/89; B01F 35/2115; B01F 35/2135; B01F 35/2207; B01F 35/2209; B01F 35/22161; B01F 2101/22

(57) ABSTRACT

A method and a system for preparing a biomaterial from at least two components, said method comprising the steps of:
providing at least two biomaterial components in a mixing compartment (3) of the preparation system (1; 1′);
measuring at least one feature in the preparation system with at least one sensor (7), said at least one feature comprising at least temperature and/or humidity;
mixing said at least two biomaterial components by a mixing device (5) of the preparation system (1; 1′) to which mixing device (5) said mixing compartment (3) can be or is connected;
controlling said mixing by a control unit (9) of the preparation system (1; 1′), said control unit (9) being in communication contact with said mixing device (5) and with said at least one sensor (7), wherein said mixing is controlled such that at least one mixing parameter of said mixing is dependent on a value of said at least one measured feature.

### TECHNICAL FIELD OF THE INVENTION

The present invention relates to a method and a system for preparing a biomaterial from at least two components.

### RELATED ART

The mixing and delivery of biomaterials in a clinical environment is challenging because of a few different circumstances. One factor is that often two or more components of the biomaterial need to be mixed shortly before the biomaterial is used. Often a delivery time window, here also called working time, after mixing of the components is very short before the biomaterial becomes unusable. Depending upon the type of biomaterial the formulation components to be mixed may often be problematic and hard to mix. If for example a calcium based cement should be mixed from a powder and a liquid, the powder may stick to surfaces and need a greater mixing energy to be completely mixed and incorporated into the liquid.

Another factor to take into account is the need for maintaining sterility up to the point of use. Furthermore, the mixed biomaterial needs to be easily and directly injectable to the site of use. The mixed biomaterial needs to meet the requirements of working time and curing time within a clinically acceptable time frame.

Many biomaterials that are mixed at the time of use have an injectability window, here also called working time, that is both temperature and time dependent and users are often confronted with a mixed paste that has become uninjectable due to exceeding this injectability window.

If a first dose of a biomaterial in the form of a cement to be used for a patient with an exposed cavity becomes uninjectable, the patient would be at additional risk of infection due to the delay while waiting to receive the second dose of cement, whilst the practitioner would have both a delay in operating schedule as well as the doubling in cost and the additional packaging disposal.

Certain calcium based cement formulations are extremely sensitive to mixing and delivery conditions and these require particular care in their mixing preparation and delivery handling.

### SUMMARY

An object of the invention is to provide an improved method and system for preparing a biomaterial from at least two components.

A further object of the invention is to provide a method and system for preparing a biomaterial from at least two components with improved mixing efficiency.

This is achieved in a method, a preparation system and a computer program according to the independent claims.

According to one aspect of the invention a method for preparing a biomaterial from at least two components in a preparation system is provided, said method comprising the steps of:
providing at least two biomaterial components in a mixing compartment of the preparation system;
measuring at least one feature in the preparation system with at least one sensor, said at least one feature comprising at least temperature and/or humidity;
mixing said at least two biomaterial components by a mixing device of the preparation system to which mixing device said mixing compartment can be or is connected;
controlling said mixing by a control unit of the preparation system, said control unit being in communication contact with said mixing device and with said at least one sensor, wherein said mixing is controlled such that at least one mixing parameter of said mixing is dependent on a value of said at least one measured feature.

According to another aspect of the invention a preparation system for preparing a biomaterial from at least two components is provided, said preparation system comprising:
a mixing compartment in which the at least two components of the biomaterial to be prepared are provided for being mixed;
a mixing device which is connected to or is connectable to the mixing compartment for inducing mixing of biomaterial provided in the mixing compartment;
at least one sensor positioned in the preparation system for measuring at least one feature, said feature comprising at least temperature and/or humidity;
a control unit which is in communication contact with said mixing device and with said at least one sensor, said control unit being configured to control said mixing device such that at least one mixing parameter of a mixing induced by the mixing device to a biomaterial provided in the mixing compartment is dependent on a value of said at least one feature measured by the at least one sensor.

According to another aspect of the invention a computer program is provided comprising computer-executable instructions which, when executed by a processor in a control unit in a preparation system as defined above, cause the control unit to control said mixing device such that at least one mixing parameter of a mixing induced by the mixing device to a biomaterial provided in the mixing compartment is dependent on a value of said at least one feature measured by the at least one sensor.

Hereby, a method and a system for preparing a biomaterial from at least two components are achieved in which a mixing of said components is controlled from a control unit and in which said mixing is controlled in dependence on at least one measured feature. Hereby mixing can be optimized for different measured features, such as for example for a measured temperature and/or a measured humidity and hereby an improved mixing efficiency is achieved. For example, different temperatures will affect different biomaterials differently. A mixing time can for example be varied in dependence of temperature and/or humidity. For some materials a decreased mixing time would be beneficial for increasing temperatures. A mixing power can also be varied in dependence of temperature and/or humidity. For some materials an increased mixing power may be needed for increased temperatures. However, for other materials it may be the opposite relation. A needed mixing energy, i.e. total mixing power during the mixing time will for some materials be decreased for increasing temperatures. For some types of biomaterial a decreased mixing time and an increased mixing power may be suitable for increasing temperatures and for other types of biomaterial, both a decreased mixing time and a decreased mixing power may be suitable for increasing temperatures. The control unit can hereby, for example be configured to control the mixing device to increase or decrease a mixing power and increase or decrease a mixing time when a measured temperature is above a certain threshold temperature. Said threshold temperature can for example be 22° C. The control unit can also be configured to control the mixing device to provide a mixing power and mixing time which is directly dependent on a measured temperature according to precalculated formulas which may be experimentally compiled.

In some embodiments of the invention said mixing is controlled such that at least one mixing parameter of said mixing also is dependent on biomaterial parameters provided to the control unit. Said biomaterial parameters can comprise information about how different mixing parameters should be changed in order for providing as good mixing as possible for a number of different types of biomaterials and in dependence of different values for said at least one measured feature. Such biomaterial parameters may have been investigated on beforehand and been preloaded into the control unit. Some biomaterial information may also be manually input by a user or digitally read into the control unit at the time of preparing the biomaterial. This can be for example information about which biomaterial is used at this specific time and amount of the different components used at this time. The control unit comprises a processor and algorithms for processing the biomaterial parameters and the at least one measured feature and for controlling the mixing device to provide an optimal mixing for this specific biomaterial under the conditions of a value of the measured at least one feature, i.e. for example at a specific measured temperature. Hereby the mixing is specifically adapted both for specific biomaterial parameters and for at least one measured feature. Hereby the mixing can be for example adapted for different temperatures which is an important factor for many biomaterials comprising at least two components, such as biomaterial cements. Hereby mixing parameters can be varied in dependence of which biomaterial that is mixed, which amount of different components that are provided in the biomaterial and in dependence of a measured feature. Hereby an efficient mixing can be provided. Mixing parameters can be for example mixing time and mixing power and if mixing is performed by vibration mixing parameters can also include amplitude and frequency of the vibrations. Values of different mixing parameters are suitably adapted differently for different materials in order to achieve as good mixing as possible. According to some examples of the method and system of the invention, where a control unit receives information about at least one measured feature and comprises or can read information about biomaterial parameters and is provided in communication contact with the mixing device such that mixing can be controlled in dependence of both biomaterial parameters and the at least one measured feature, such a controlled mixing is possible. Hereby mixing can be optimized for different factors and an improved mixing efficiency is achieved. Furthermore, there is a lower risk for the biomaterial turning hard and uninjectable before it is being used.

In one embodiment of the invention the method further comprises the step of delivering said mixed biomaterial directly from said mixing compartment to a location where it should be used. In such an embodiment said mixing compartment comprises a delivery part through which the mixed biomaterial can be delivered directly to a location where it should be used. Hereby a closed and aseptic handling of the biomaterial all the way through preparing and delivering can be assured.

In one embodiment of the invention the method further comprises a step of sending an alert from the control unit to a user of the preparation system when the mixing is ready, said alert optionally also being indicative of for how long time the mixed biomaterial is usable, i.e. a working time of the mixed biomaterial. In such an embodiment the control unit is configured for sending an alert to a user of the preparation system. Hereby the user will get aware of how long time there is before the biomaterial is not usable anymore and hereby waste of material can be avoided.

In one embodiment of the invention the method further comprises a step of sending a warning from the control unit to a user of the preparation system if a measured temperature is over a certain threshold temperature over which temperature the biomaterial to be prepared is not suitable to prepare. Hereby a user can take actions, such as for example cooling down the biomaterial before preparation and hereby possible waste of material can be avoided.

In one embodiment of the invention said mixing compartment is a closed and sterile or aseptic mixing compartment. Hereby an aseptic handling of the biomaterial can be assured.

In one embodiment of the invention at least the mixing device, the mixing compartment and the at least one sensor are combined in one handheld device which is also used for delivering the prepared biomaterial to a wanted location.

In one embodiment of the invention the preparation system further comprises a display, signal lights or a speaker being in communication contact with the control unit and being configured for presenting information relating to the preparation process from the control unit to a user of the preparation system.

Further embodiments are described in the dependent claims and in the detailed description.

### BRIEF DESCRIPTION OF THE DRAWINGS

FIG. 1a is a schematic drawing of a preparation system according to one example of the invention.

FIG. 1b is a schematic drawing of a preparation system according to another example of the invention.

FIG. 2 is a flow chart of a method according to one example of the invention.

### DETAILED DESCRIPTION

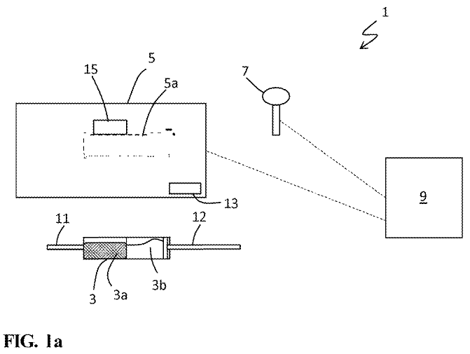

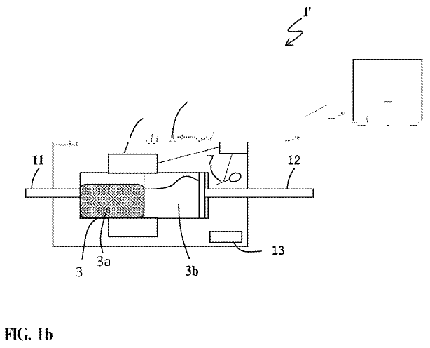

A preparation system 1 for preparing a biomaterial from at least two components according to one example of the invention is schematically shown in FIG. 1a. Another example of a preparation system 1′ according to the invention is schematically shown in FIG. 1b. The two examples are similar and similar parts are given the same reference numbers. The difference between these two examples is that in the preparation system 1′ of FIG. 1b all components are provided in one and the same handheld device 6, possibly except for a control unit 9 which can be provided either inside the handheld device 6 or remote from the handheld device. In the preparation device 1 as shown in FIG. 1 the different parts can be provided separate which will be described in more detail below. The two examples are described together below.

Biomaterials which are prepared from at least two components are for example different types of cement. Such as for example calcium based cements, such as hydroset, tetranite and Osstic.

The two or more components of these types of materials need to be mixed at the time of use. The components can for example be one in liquid form and one in powder form.

The preparation system 1; 1′ comprises a mixing compartment 3 in which the at least two components of the biomaterial to be prepared are provided for being mixed. The mixing compartment 3 can in some examples be a closed and sterile or aseptic compartment. This is suitable because many types of biomaterials need to be kept sterile until delivery to the place of use. Suitably the biomaterial can also be directly delivered out from the mixing compartment 3 to the position where it should be used. For this purpose, the mixing compartment comprises in some examples a delivery part 11 through which the biomaterial can be delivered directly to a location where it should be used. The mixing compartment 3 may furthermore comprise some kind of piston or plunger 12 for pushing out the biomaterial via the delivery part 11. However, in some embodiments the mixing compartment 3 can instead be flexible whereby the biomaterial can be pushed out from the mixing compartment 3 via the delivery part 11 by squeezing the mixing compartment 3.

The at least two components can be preloaded in the mixing compartment 3. However, in another example of the invention a user can load the components himself into the mixing compartment. In some examples the mixing compartment comprises two or more chambers 3a, 3b. The two or more chambers 3a, 3b are separated by some kind of wall 4. This may be for example a breakable wall or an openable wall. Such a wall 4 can for example be broken or opened during an initial phase of mixing. Mixing will be provided to the mixing chamber 3 by a mixing device 5 which will be further described below. Hereby the at least two components can be mixed when the wall 4 has been broken or opened. When the at least two components are preloaded in the mixing compartment 3 such separate chambers 3a, 3b are suitable because otherwise the components would start mix before the time for use and that would often destroy the biomaterial. This type of biomaterial needs to be used almost directly after its components have been mixed as discussed above. Two or more separate chambers 3a, 3b can be provided in the mixing compartment 3 in different ways. In FIGS. 1a and 1b two serial chambers 3a, 3b are shown. However, the chambers can as well be provided in parallel. Other possibilities than a wall 4 between the chambers can also be provided, such as breakable capsules which are separately enclosing the at least two components and which can break during an initial phase of a mixing process. The whole mixing compartment 3 is in some examples of the invention disposable.

The preparation system 1; 1′ comprises furthermore a mixing device 5 which is connected to or is connectable to the mixing compartment 3 for inducing mixing of biomaterial provided in the mixing compartment 3. In the preparation system 1 as shown in FIG. 1a the mixing device 5 is a separate part to which the mixing compartment 3 can be connected. Hereby the mixing compartment 3 which is comprising at least two components to be mixed, can be connected to the mixing device 5 when the biomaterial should be prepared. A connection position 5a is schematically shown in the mixing device 5 in FIG. 1a for illustrating where the mixing compartment can be connected. When mixing of the components is ready the mixing compartment 3 can be released and suitably also directly used for delivery of the mixed biomaterial.

In FIG. 1b instead a handheld device 6 is shown comprising both the mixing device 5 and the mixing compartment 3. The mixing device 5 is connected to the mixing compartment 3.

In some examples the whole handheld device 6 can be a disposable device. However, in some examples the mixing compartment 3 can be exchangeable whereby in such examples only the mixing compartment is disposable and the rest of the handheld device 6 can be reused for another mixing compartment 3. The handheld device 6 is suitably also used for delivery of the mixed biomaterial.

In both the examples as shown in FIGS. 1a and 1b the mixing can be performed for example by vibrating the mixing compartment 3 by the mixing device 5 or by inducing vibration in the biomaterial components by the mixing device 5 by ultrasound. Hereby the mixing device 5 comprises some kind of excitation device 15 which can be for example a vibration device and/or an ultrasonic device whereby vibrations or ultrasound can be transferred to the mixing compartment 3 when the mixing compartment 3 is connected to the mixing device 5. The excitation device 15 can in another embodiment comprise another means of mechanically transferring a mixing power to the mixing compartment, such as for example movement of an agitation member.

The preparation system 1; 1′ comprises furthermore at least one sensor 7 positioned in the preparation system 1; 1′ for measuring at least one feature, said feature comprising at least temperature and/or humidity. The sensor 7 can be positioned anywhere nearby the mixing compartment 3 or inside the mixing compartment 3. In the example as shown in FIG. 1b the handheld device 6 can for example comprise the at least one sensor 7. More than one sensor can be provided and more than one feature can be measured.

The preparation system 1; 1′ comprises furthermore a control unit 9 which is in communication contact with said mixing device 5 and with said at least one sensor 7. The control unit 9 can be a separate unit or can be provided inside the mixing device 5 or inside the handheld device 6. The control unit can be provided in a mobile telephone or a computer or any other device and comprises a processor for executing instructions of a computer program for controlling the mixing device 5. Said control unit 9 is configured to control said mixing device 5 such that at least one mixing parameter of a mixing induced by the mixing device 5 to a biomaterial provided in the mixing compartment 3 is dependent on a value of said at least one feature measured by the at least one sensor 7. Said mixing parameters can also be controlled to vary during the time of mixing. A mixing may comprise different phases which will be described in more detail below. Said mixing parameters can comprise one or more of: time of mixing, power of mixing, amplitude of vibration or ultrasound and frequency of vibration or ultrasound. Hereby a mixing can be performed in dependence on at least one measured feature, i.e. at least one mixing parameter will vary in dependence of a value of a measured feature.

If for example the feature is temperature and a mixing parameter is power of mixing, the power of mixing can, in some examples of the invention and for some materials be increased when a temperature is increasing. For other materials the power of mixing can instead be decreased when a temperature is increasing. If also the time of mixing is a mixing parameter to be controlled the time of mixing can in some examples of the invention be decreased when a temperature is increasing if at the same time the power of mixing is increased. For some materials it would be beneficial to provide a mixing which is shorter in time but having a higher mixing power when a temperature is increasing. However, for other types of material it may be suitable to both decrease mixing time and mixing power when temperature is increasing. Different materials will behave differently at different temperatures and both flow behaviour and solubility of the different components to be mixed will affect the mixing process. Hereby experimental data for each biomaterial to mix can suitably be used for compiling the algorithms to be used by the control system 9 for controlling of the mixing. A similar control of mixing parameters can be provided in dependence of a measured humidity. The control of the mixing can be stepless, i.e. directly dependent on a measured temperature or humidity. Alternatively, the mixing can be controlled differently for different predefined temperature ranges. The mixing parameters can for example be changed if the temperature increases over a certain threshold temperature, such as for example above 22° C. Mixing parameters may also comprise frequency and/or amplitude of vibrations for the mixing. Hereby also frequency and/or amplitude can in some examples be varied for improving mixing. In some examples frequency can be increased for increased temperatures in order to provide efficient mixing.

However, in other examples frequency and/or amplitude may be varied differently. Suitable frequencies and amplitudes for different temperatures and/or humidity can be achieved from experimental studies for the different biomaterials.

For example, the formulation design of calcium-based biomaterials which are designed for injection usually targets a range of ambient temperature that is between 18 and 22° C. When the temperature is in the range 22 to 26° C. the biomaterial is still mixable but would require a reduced mixing time and an increased mixing frequency because there is a shorter time to accomplish the mixing before the material will begin to thicken and become uninjectable.

Algorithms in a computer program which is executed in the control unit for controlling the mixing performed by the mixing device 5 can be designed from experiments for different biomaterials. Mixing time, mixing power, frequency and/or amplitude of vibrations may be varied according to different optimal schemes for different materials which may be found by experiments. Different types of biomaterials which are prepared from at least two components according to the invention will behave differently in dependence of temperature. There may be materials which need increased mixing power for increased temperatures and there may be materials which need decreased mixing power for increased temperatures. The mixing time needs also to be adjusted for increasing temperatures. For some materials which are curing faster at higher temperatures a decreased mixing time is suitable when temperature is increasing. If mixing is performed by vibration also the frequency and amplitude can be varied for providing optimal mixing. Optimal frequency and amplitude for different materials may be found by experiments. A higher frequency may be suitable for increasing temperatures in order to avoid curing of the material.

Another important factor for these types of calcium-based biomaterials and also for other types of biomaterials which are prepared from at least two components, is the working time before the material becomes uninjectable. The biomaterial needs to be injected or used within this working time. Setting time or curing time is a time until the biomaterial for example is tap hard. Both working time and setting time are temperature dependent, whereby increased ambient temperature often will reduce both working and setting times whilst decreased temperature will increase these times. When the ambient temperature is in the range 22-26° C. the working time will be reduced by up to 50% in comparison to the working time in the temperature range of 18-22° C. for some materials. Alerts and warnings related to for example working time and too high temperatures can be sent to a user of the preparation device which will be further discussed below. The preparation system can for example warn the user to refrigerate the biomaterial for a time before preparation if the temperature is above a certain temperature. For some biomaterials such a warning can be sent for temperatures above for example 26° C.

The mixing device 5 may in some examples, as mentioned above, comprise some kind of excitation device 15 which can be for example a vibration device or an ultrasonic device. Hereby the mixing parameters to be varied may not only be time and power but may also include amplitude and frequency. Depending on which type of excitation device is used the possible amplitude and frequency ranges will differ. One example of an excitation device 15 is a piezo electric vibration element which would have a relatively low amplitude and high frequency. An ultrasonic device, such as an ultrasonic horn, will have a lower amplitude and an ultra-high frequency.

The control system 9 can hereby be configured to control the mixing device 5 to provide mixing by vibrations of a certain amplitude and/or frequency in dependence of a measured feature, for example a measured temperature or humidity. A higher frequency and/or amplitude of vibrations for mixing may for example be suitable for higher temperatures. The frequency and/or amplitude may for example be increased or decreased with increased temperature.

The mixing process, also called mixing cycle, can furthermore be controlled in more detail.

The mixing parameters may in some examples of the invention suitably be controlled to be different in different phases of the mixing process. For example, there may be a first phase of mixing requiring higher energy than a second phase of the mixing. The mixing of powders and liquid in general requires different frequencies and amplitudes during the mixing cycle. In a first phase the liquid and power may require a larger amplitude and a lower frequency, compared to amplitudes and frequencies for following phases, to begin incorporation of the liquid in the powder mass. When the first phase is completed, i.e. there is no large pockets of dry powder left, a lower amplitude and a higher frequency is required until powder clumps are incorporated. There may also be a third phase where the lower amplitude may be maintained and the frequency may be reduced to complete the mixing, whereby a completely smooth homogenous paste has been achieved. As an illustrative example amplitude and frequency can be varied in the different phases as follows: For a first phase the amplitude can be chosen to be equal to a maximum amplitude and the frequency can be chosen to be 20% of a maximum frequency. For a second phase the amplitude can be chosen to be 50% of the maximum amplitude and the frequency can be 80% of a maximum frequency and in a third phase amplitude can be chosen to be 20% of a maximum amplitude and frequency can be 20% of a maximum frequency. The time duration for each of the phases can of course be varied differently as required by different materials. The percentage numbers given above are only examples and may also be varied differently for different materials.

Hereby a control of the mixing can comprise many variables and the mixing can be made very specific for different materials and different circumstances, such as ambient temperature and/or humidity. Hereby the control unit comprises all such information about different mixing phases and different mixing parameters for different biomaterials for the different mixing phases. This information is included in the biomaterial parameters which will be further described below. The control unit is configured for controlling the mixing device accordingly.

In some examples of the invention said control unit 9 is further configured to control said mixing device 5 such that at least one mixing parameter of said mixing is also dependent on biomaterial parameters provided to the control unit 9. The biomaterial parameters can be preloaded in the control unit 9 or can be input to the control unit. Input can be manual input by a user of the preparation system or digital reading from, for example a tag. Such a tag can for example be provided to the mixing compartment 3 and can comprise information about type of material and amount of the different components.

Said biomaterial parameters may comprise information about how different mixing parameters suitably are changed for providing a good mixing for a number of different types of biomaterials and in dependence of different values for said at least one measured feature. This can for example be information about how long mixing time and/or which mixing power (possibly including vibration frequency and/or amplitude) is needed for a certain material at a certain temperature and/or humidity. Said biomaterial parameters comprise suitably also information about type of biomaterial and amount of one or more of the components of the biomaterial.

The mixing parameters can also be varied throughout a process for mixing a biomaterial. Hereby the control unit comprises information about such variations such that the mixing device can be controlled from the control unit to vary the mixing parameters during mixing according to the specific biomaterial being mixed. For example, it may be suitable to start at a slow mixing frequency and increase the mixing frequency during mixing. This was described above as different phases of the mixing. Hereby all necessary information for controlling the mixing device 5 in an optimal way for different types of biomaterials and at different ambient conditions are provided in the control unit.

In some examples of the invention the control unit 9 is configured for sending an alert to a user of the preparation system 1; 1′ when the mixing is ready. Said alert optionally also being indicative of for how long time the mixed biomaterial is usable, i.e. the working time for the mixed biomaterial before it becomes uninjectable. The time window for usability of the mixed biomaterial, i.e. working time, is in some examples dependent on the measured at least one feature. For example, a high temperature, for example above 22° C. for some biomaterials, would decrease the working time. Above a certain temperature, for example above 26° C. for some materials, the biomaterial is not usable at all. The alert can be in the form of a visible or audible signal. A visible signal can for example be a light, possibly a light with different colours. In that case a red light can be activated when the biomaterial is not usable anymore. A green light could be shown when mixing is ready and for example a yellow light when only a short period is left of a working time window for usability, for example 10 seconds, before the red light will be activated. Of course, there are many other similar alert systems which can as well be used. For example, a display 13 can be provided somewhere in the preparation system 1; 1′. On such a display any information can be shown, such as information that mixing is ready, information about for how long time the mixed biomaterial will be useful, a timer counting down the working time window for usability, information showing that the biomaterial is not usable any longer when the time window has elapsed etc. The alert could also be as an audible signal. In that case a speaker is provided somewhere in the preparation system. The audible signal can comprise information about when the mixing is ready and a working time window of usability etc. The audible information can be spoken words or sounds indicating end of time window, such as increasing beeping.

In some examples of the invention the control unit 9 is configured for sending a warning from the control unit 9 to a user of the preparation system if a measured temperature is over a certain threshold temperature over which temperature the biomaterial to be prepared is not suitable to prepare.

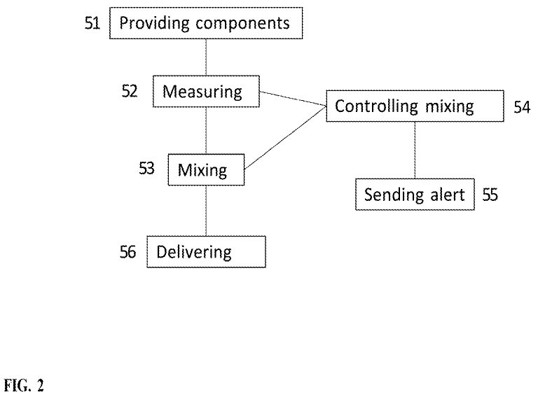

In FIG. 2 a flow chart of a method for preparing a biomaterial from at least two components in a preparation system as described above in relation to FIGS. 1a and 1b, according to one example of the invention is shown. The method steps are described in order below:

S1: Providing at least two biomaterial components in a mixing compartment 3 of the preparation system 1; 1′.

S2: Measuring at least one feature in the preparation system with at least one sensor 7, said feature comprising at least temperature and/or humidity. The at least one feature is measured for example in ambient air surrounding the mixing compartment 3 in the preparation system or possibly directly inside the mixing compartment 3.

S3: Mixing said at least two biomaterial components by a mixing device 5 of the preparation system 1; 1′ to which said mixing compartment 3 can be or is connected.

S4: Controlling said mixing by a control unit 9 of the preparation system 1; 1′, said control unit 9 being in communication contact with said mixing device 5 and with said at least one sensor 7. The step of controlling the mixing, S4, is hereby not performed after the step of mixing, S3, which is illustrated in FIG. 2 by placing the step of controlling the mixing, S4, at the side. Said mixing is hereby controlled such that at least one mixing parameter of said mixing is dependent on a value of said at least one measured feature. In some examples said mixing is further controlled such that at least one mixing parameter of said mixing also is dependent on biomaterial parameters provided to the control unit 9. Mixing parameters and biomaterial parameters have been discussed above.

The method may also comprise a further step of:

S5: Sending an alert from the control unit 9 to a user of the preparation system 1; 1′ when the mixing is ready, said alert optionally also comprising information about for how long time the mixed biomaterial is usable. In some examples a warning may also be sent to user if a temperature is measured to be above a threshold temperature where it is not suitable to prepare the biomaterial as discussed above. Such alert and warnings can be sent by different means as discussed above, for example as visible or audible signals or as texts on a display. The two steps S4 and S5: “controlling mixing” and “sending alert” are performed by the control unit 9 and are illustrated in FIG. 2 at the side of the other method steps.

The method may also comprise a further step of:

S6: Delivering said mixed biomaterial directly from said mixing compartment 3 to a location where it should be used.

A computer program is also provided according to the invention. Said computer program comprises computer-executable instructions which, when executed by a processor in a control unit 9 in a preparation system 1; 1′ as described above, cause the control unit 9 to control said mixing device 5 such that at least one mixing parameter of a mixing induced by the mixing device 5 to a biomaterial provided in the mixing compartment 3 is dependent on a value of said at least one feature measured by the at least one sensor 7.

A computer program product comprising a computer-readable storage medium is also provided, the computer-readable storage medium having the computer program as defined above embodied therein.

In one example a method for preparing a biomaterial from at least two components in a preparation system is provided. Said method comprising the steps of:
providing at least two biomaterial components in a mixing compartment of the preparation system;
measuring at least one feature in the preparation system with at least one sensor, said at least one feature comprising at least temperature and/or humidity;
mixing said at least two biomaterial components by a mixing device of the preparation system to which said mixing compartment can be or is connected;
sending an alert to a user of the preparation system from a control unit provided in the preparation system, said control unit being in communication contact with said at least one sensor, said alert comprising information about a working time window during which the mixed biomaterial is usable and said information being prepared by the control unit in dependence of said at least one measured feature.

Examples of how alerts or warnings can be sent to a user has been discussed above, i.e. by light, sound or information on a display.

1. A method for preparing a biomaterial from at least two components in a preparation system (1; 1′), said method comprising the steps of:
providing at least two biomaterial components in a mixing compartment (3) of the preparation system (1; 1′);
measuring at least one feature in the preparation system with at least one sensor (7), said at least one feature comprising at least temperature and/or humidity;
mixing said at least two biomaterial components by a mixing device (5) of the preparation system (1; 1′) to which mixing device (5) said mixing compartment (3) can be or is connected;
controlling said mixing by a control unit (9) of the preparation system (1; 1′), said control unit (9) being in communication contact with said mixing device (5) and with said at least one sensor (7), wherein said mixing is controlled such that at least one mixing parameter of said mixing is dependent on a value of said at least one measured feature.

2. The method according to claim 1, wherein said mixing is controlled such that at least one mixing parameter of said mixing also is dependent on biomaterial parameters provided to the control unit (9).

3. The method according to claim 2, wherein said biomaterial parameters comprise
(a) information about how different mixing parameters are suitably changed for providing a good mixing for a number of different types of biomaterials and in dependence of different values for said at least one measured feature or
(b) information about type of biomaterial and amount of one or more of the components of the biomaterial.

4. (canceled)

5. The method according to claim 1, wherein said at least one mixing parameter comprises time of mixing and/or power of mixing.

6. The method according to claim 1, wherein said mixing comprises vibrating the mixing compartment (3) by the mixing device (5) or inducing vibration in the biomaterial components by the mixing device (5) by ultrasound, wherein said mixing parameters can comprise one or more of: time of mixing, power of mixing, amplitude of vibration or ultrasound and frequency of vibration or ultrasound.

7. The method according to claim 1, further comprising the step of delivering said mixed biomaterial directly from said mixing compartment (3) to a location where it should be used.

8. The method according to claim 1, further comprising a step of sending an alert from the control unit (9) to a user of the preparation system (1; 1′) when the mixing is ready, said alert optionally also being indicative of for how long time the mixed biomaterial is usable.

9. The method according to claim 1, further comprising a step of sending a warning from the control unit (9) to a user of the preparation system if a measured temperature is over a certain threshold temperature over which temperature the biomaterial to be prepared is not suitable to prepare.

10. A preparation system for preparing a biomaterial from at least two components, said preparation system (1; 1′) comprising:
a mixing compartment (3) in which the at least two components of the biomaterial to be prepared are provided for being mixed;
a mixing device (5) which is connected to or is connectable to the mixing compartment (3) for inducing mixing of biomaterial provided in the mixing compartment (3);
at least one sensor (7) positioned in the preparation system (1; 1′) for measuring at least one feature, said feature comprising at least temperature and/or humidity;
a control unit (9) which is in communication contact with said mixing device (5) and with said at least one sensor (7), said control unit (9) being configured to control said mixing device (5) such that at least one mixing parameter of a mixing induced by the mixing device to a biomaterial provided in the mixing compartment is dependent on a value of said at least one feature measured by the at least one sensor (7).

11. The preparation system according to claim 10, wherein said control unit (9) is further configured to control said mixing device (5) such that at least one mixing parameter of said mixing also is dependent on biomaterial parameters provided to the control unit (9).

12. The preparation system according to claim 10, wherein said biomaterial parameters comprise information about
(a) how different mixing parameters are suitably changed for providing a good mixing for a number of different types of biomaterials and in dependence of different values for said at least one measured feature, or
(b) type of biomaterial and amount of one or more of the components of the biomaterial.

13. (canceled)

14. The preparation system according to claim 10, wherein said at least one mixing parameter comprises time of mixing and/or power of mixing.

15. The preparation system according to claim 10, wherein said mixing device (5) is configured for vibrating the mixing compartment (3) or inducing vibration in the biomaterial components by the mixing device (5) by ultrasound, wherein said mixing parameters can comprise one or more of: time of mixing, energy of mixing, amplitude of vibration or ultrasound and frequency of vibration or ultrasound.

16. The preparation system according to claim 10, wherein said mixing compartment (3)
(a) is a closed and sterile or aseptic mixing compartment, or
(b) comprises a delivery part (11) through which the mixed biomaterial can be delivered directly to a location where it should be used.

17. (canceled)

18. The preparation system according to claim 10, wherein the control unit (9) is configured for sending an alert to a user of the preparation system (1; 1′) when the mixing is ready, said alert optionally also comprising information about for how long time the mixed biomaterial is usable.

19. The preparation system according to claim 10, wherein the control unit (9) is configured for sending a warning to a user of the preparation system if a measured temperature is over a certain threshold temperature over which temperature the biomaterial to be prepared is not suitable to prepare.

20. The preparation system according to claim 10, wherein at least the mixing device (5), the mixing compartment (3) and the at least one sensor (7) are combined in one handheld device (6) which is also used for delivering the prepared biomaterial to a wanted location.

21. The preparation system according to claim 10, further comprising a display (13), signal lights or a speaker being in communication contact with the control unit (9) and being configured for presenting information relating to the preparation process from the control unit to a user of the preparation system.

22. A computer program comprising computer-executable instructions which, when executed by a processor in a control unit (9) in a preparation system (1; 1′) according to claim 10, cause the control unit (9) to control said mixing device (5) such that at least one mixing parameter of a mixing induced by the mixing device (5) to a biomaterial provided in the mixing compartment (3) is dependent on a value of said at least one feature measured by the at least one sensor (7).

23. A computer program product comprising a computer-readable storage medium, the computer-readable storage medium having the computer program according to claim 22 embodied therein.
